(19) United States
(12) Patent Application Publication
(10) Pub. No.: US 2025/0366864 A1
(43) Pub. Date: Dec. 4, 2025

(54) Bone Resection Template

(71) Applicant: Howmedica Osteonics Corp., Mahwah, NJ (US)

(72) Inventors: Keith Chakramakkil, Mumbai (IN); Kenneth Pascale, Hoboken, NJ (US)

(21) Appl. No.: 19/225,664

(22) Filed: Jun. 2, 2025

Related U.S. Application Data
(60) Provisional application No. 63/655,197, filed on Jun. 3, 2024.

(51) Int. Cl. A61B 17/15; A61B 17/00

(52) U.S. Cl. CPC A61B 17/155; A61B 2017/0042; A61B 2017/00982

(57) ABSTRACT

A bone resection template includes a plate body and an extension arm extending from the plate body. The plate body has a length extending from a first end to a second end opposite the first end and the extension arm protrudes from the plate body proximate the second end. The extension arm includes a portion with a curved surface contoured to receive a bone therein such that when the bone is received on the curved surface portion the plate body is on one side of the bone.

### CROSS-REFERENCE TO RELATED APPLICATIONS

This application claims the benefit of the filing date of U.S. Provisional Patent Application No. 63/655,197 filed on Jun. 3, 2024, the disclosure of which is hereby incorporated by reference.

### BACKGROUND

When a bone must be resected due to bone loss, a tumor, or other deficiency, surgical instrumentation may be used to aid in the identification of the proper resection location on the bone. One such instrument is a bone resection template. For example, femoral bone resection templates may be shaped to rely on a silhouette of a distal condyle of a femur to determine patella tracking and the joint line between the femur and the tibia. The template is aligned with the condyle silhouette and an elongate part of the template may be compared with a corresponding location on the femur to indicate the resection level along the femur. However, in instances where there is severe bone loss or the bone features are not visible, it is difficult to use the distal femur or other nearby bone features as landmarks for measuring the bone cut. Thus, identification of a patella position may rely on a coarse approximation of a distal femur or other bone, thereby making the measurement process difficult.

Accordingly, a need exists to develop a bone resection template that can measure the bone resection level in cases of severe bone loss or deformities that does not require a healthy bone as a reference.

### BRIEF SUMMARY

The present disclosure addresses the need for improved bone templates by providing a bone resection template that measures a femoral resection level using an inferior pole of a patella as a guide. The bone resection template includes a plate body having an extension arm that is contoured to grasp the patella while the plate body is positionable against a distal femur. The plate body further includes indicia that allow for determination of a resection level of the distal femur using the inferior pole of the patella as a reference. The bone templates according to the present disclosure provide surgeons with the ability to measure accurate resection levels in cases of severe bone loss, deformity, or other deficiencies.

In a first aspect, the present disclosure relates to a bone resection template. In one embodiment, the bone resection template includes a plate body having a length extending from a first end to a second end opposite the first end and an extension arm protruding from an end portion of the plate body proximate the second end. The extension arm including a portion with a curved surface contoured to receive a bone therein such that when the bone is received on the curved surface portion, the plate body is on one side of the bone.

In one embodiment, the plate body includes an elongate portion and the end portion, the end portion being wider than the elongate portion, and the extension arm extending from a lateral side of the end portion. The extension arm further includes a transition portion and a gripping portion. The transition portion defines a space that separates the gripping portion of the extension arm from the plate body. The gripping portion of the extension arm includes a neck portion between the transition portion and a free end of the extension arm. The neck portion of the extension arm is defined by a first curved surface and a second curved surface opposite the first curved surface. The first curved surface and the second curved surface are concave.

In some examples, a length of the extension arm may be less than a length of the end portion of the plate body. Optionally, a length of the extension arm may be equal to a length of the end portion of the plate body.

In other examples, the plate body may include a plurality of indicia along a direction between the first end and the second end, the indicia indicative of a dimension from an end of a bone measured by the bone template. The plurality of indicia are a plurality of notches along one or more edges of the plate body. Optionally, the extension arm may include at least one slot.

In some examples, the end portion of the plate body may include indicia representative of a location of a bone-gripping edge of the extension arm along a direction of the length of the plate body. The indicia may be a linear marking extending across the width of the end portion on a surface of the end portion facing away from the extension arm. The linear marking may be positioned in a range from 10 mm to 20 mm from the second end of the plate body.

In other examples, the plate body includes an elongate portion and a transition portion that connects the elongate portion of the plate body to the extension arm. The extension arm includes a base portion and a gripping portion positioned to form a trough within the extension arm. The gripping portion includes a curved surface, the curved surface having a plurality of teeth protruding from the curved surface of the gripping portion.

In some examples, the elongate portion of the plate body may include a plurality of indicia along a direction of the length of the plate body. The plurality of indicia may be a plurality of notches.

In one example, the extension arm extends from a single end of the plate body, the extension arm having a length from the plate body to a free end. Optionally, the plate body may include a first elongate portion and a second elongate portion, the extension arm separating the first and second elongate portions.

In a second aspect, the present disclosure relates to a device for measuring a bone cut. In one embodiment, a device for measuring a bone cut includes an elongate member, extending from a first end portion to a second end portion opposite the first end portion, and a gripping member extending from the second end portion. The elongate member includes a measurement portion with indicia indicating a dimension based on a size of the device. The gripping member has a width dimension such that the gripping member includes a portion that extends laterally away from a central longitudinal axis of the measurement portion of the elongate member. The gripping member is shaped to be positionable against a bone surface such that the bone is at least partially in between the gripping member and the elongate member.

In some examples, the gripping member includes a concave edge positionable against the bone surface. The second end portion includes a curved edge positionable against a second bone surface. The indicia may include notches along the measurement portion.

In other examples, the elongate member and the gripping member are connected via a curved member on the gripping member that extends from the gripping member to the elongate member. The gripping member includes a plurality of pointed protrusions extending from a concave edge of the gripping member. Optionally, the elongate member includes a second measuring portion and a second curved member.

In a third aspect, the present disclosure relates to a method for determining an extent of a bone cut from a length of a bone. The method includes aligning a plate body of a bone resection template with an orientation of an elongate dimension of a first bone; positioning a contoured surface on an extension arm of the bone resection template onto a second bone, the extension arm protruding from the plate body of the bone resection template; and identifying a desired measurement of the first bone based on indicia on the plate body, the desired measurement representing a dimension of a portion of the first bone from an end of the first bone proximate the extension arm to the indicia.

In some examples, the method includes moving the second bone laterally to gain exposure to the first bone; and after aligning the plate body, moving the second bone to engage the extension arm of the plate body.

In some examples, the method includes moving soft tissue surrounding the first bone to position the plate body directly on the surface of the first bone.

In some examples, aligning the plate body with the first bone further comprises aligning a bottom edge of the plate body with a bottom edge of the first bone.

In some examples, the method includes fixing an edge of the extension arm around an end of the second bone such that the plate body is adjacent soft tissue extending from the second bone, the second bone being adjacent to the first bone.

In some examples, the method includes gripping an end of the second bone with a plurality of pointed protrusions extending from the extension arm.

In some examples, identifying the desired measurement of the first bone includes determining at least one resection level along the first bone via slots spaced apart along the plate body.

In other examples, the method includes determining at least one resection level along the first bone by referencing the first bone using a reference plate configured to be inserted horizontally into a slot on the plate body.

In some examples, the method includes referencing a linear marking on the plate body proximate to the extension arm to identify a distal end of the first bone.

### BRIEF DESCRIPTION OF THE DRAWINGS

A more complete appreciation of the subject matter of the present disclosure and the various advantages thereof may be realized by reference to the following detailed description which refers to the accompanying drawings, in which:

FIG. 1A is a front perspective view of a bone resection template in accordance with one embodiment of the present disclosure.

FIG. 1B is a back perspective view of the bone resection template of FIG. 1A.

FIG. 1C is a diagram of a side view of the bone resection template of FIG. 1A placed against soft tissue adjacent to the femur.

FIG. 2A is a front perspective view of a bone resection template in accordance with another embodiment of the present disclosure.

FIG. 2B is a back perspective view of the bone resection template of FIG. 2A.

FIG. 3A is a front perspective view of a bone resection template in accordance with yet another embodiment of the present disclosure.

FIG. 3B is a perspective view of the bone resection template of FIG. 3A placed against a distal portion of the femur.

FIG. 4A is a front perspective view of a bone resection template in accordance with yet another embodiment of the present disclosure.

FIG. 4B is a side view of the bone resection template of FIG. 4A placed against a distal portion of the femur and engaging a patella.

FIG. 5A is a front perspective view of a bone resection template in accordance with yet another embodiment of the present disclosure.

FIG. 5B is a side view of the bone resection template of FIG. 5A placed against a distal portion of the femur and engaging the patella.

FIG. 6 is a chart illustrating a method of determining an extent of a bone cut from a length of a bone according to one embodiment of the present disclosure.

### DETAILED DESCRIPTION

To aid the Patent Office and any readers of any patent issued on this application in interpreting the claims appended hereto, Applicant notes that it does not intend any of the appended claims or claim elements to invoke 35 U.S.C. § 112(f) unless the words “means for” or “step for” are explicitly used in the particular claim.

In describing preferred embodiments of the disclosure, reference will be made to directional nomenclature used in describing the human body. It is noted that this nomenclature is used only for convenience and that it is not intended to be limiting with respect to the scope of the present disclosure. As used herein unless stated otherwise, the term “proximal” means closer to the heart and the term “distal” means further from the heart. The term “anterior” means toward the front part of the body, and the term “posterior” means toward the back part of the body. The term “medial” means closer to or toward the midline of the body, and the term “lateral” means further from or away from the midline of the body. The term “inferior” means closer to or toward the feet, and the term “superior” means closer to or toward the crown of the head. In addition, the terms “about,” “generally,” and “substantially” are intended to mean that slight deviations from absolute are included within the scope of the term so modified.

In one aspect, the present disclosure relates to a device for identifying and guiding a bone cut. Such device may be a bone resection template. Examples of such bone resection templates are shown in the figures. In some examples, the bone resection template may be of a unitary design and may be formed of a singular plate body including an extension arm protruding from the plate body. In some examples, the plate body may include an extension arm having a gripping portion with a curved surface contoured to receive a bone. In other examples, the plate body may include an extension arm having a gripping portion with a curved surface having a plurality of teeth configured to engage the bone. The bone resection template may be designed for use as part of a surgery to replace a distal femur. The bone resection template may be used during a distal femur resection to guide the resection level of the femur using a first bone as a reference, as described in greater detail in the method of the present disclosure.

Reference will now be made in detail to the various embodiments of the present disclosure illustrated in the accompanying drawings. Wherever possible, the same or like reference numbers will be used throughout the drawings to refer to the same or like features within a different series of numbers (e.g., 100-series, 200-series, etc.). The differences between different embodiments will be focused on below, with the understanding that portions of the bone resection templates may be similar or even identical between the different embodiments.

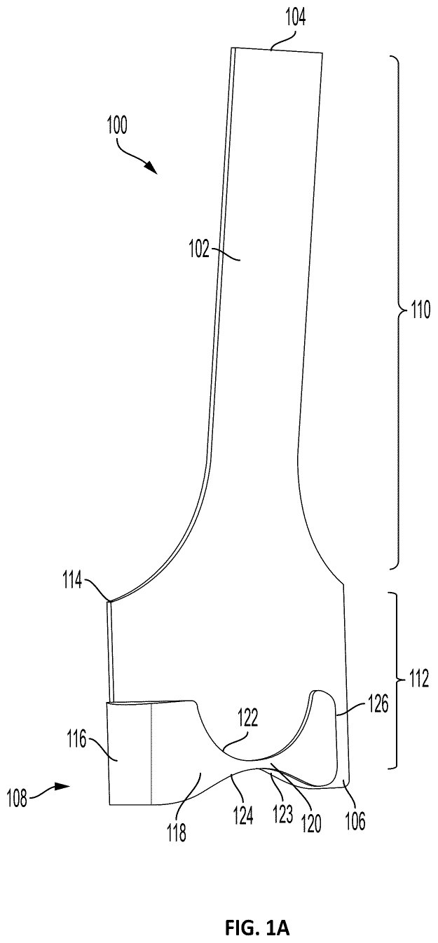

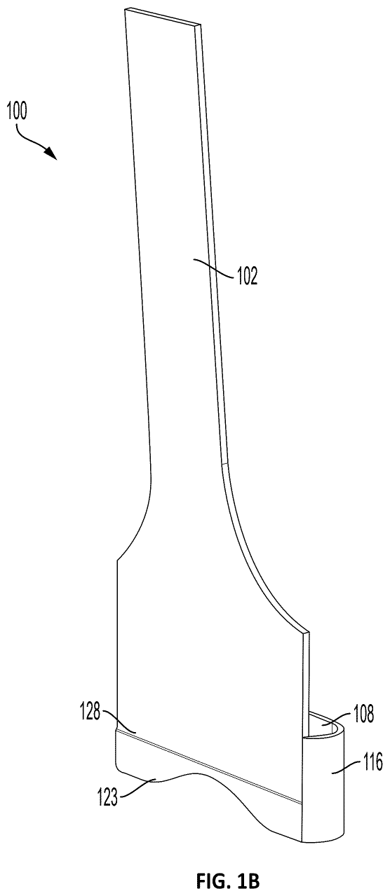

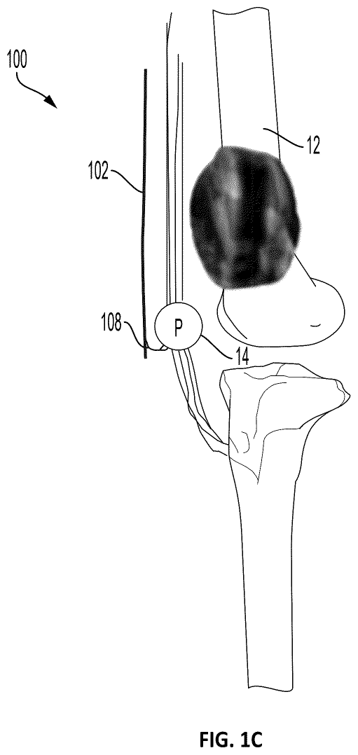

In one embodiment, as shown in FIGS. 1A and 1B, the bone resection template 100 includes a plate body 102 extending from a first end 104 to a second end 106. Plate body 102 includes an extension arm 108 extending from plate body. Plate body 102 also includes an elongate portion 110 and an end portion 112. Elongate portion 110 extends from a first end 104 of plate body 102 to a transition location 114. Transition location 114 is located at a transition point between elongate portion 110 and end portion 112 of plate body 102. End portion 112 extends from transition location 114 to a second end 106 of plate body 102. As shown in FIGS. 1A and 1B, end portion 112 has a length less than a length of the elongate portion 110 but a length greater than a length of the extension arm 108. Further, end portion 112 is wider than elongate portion 110. Elongate portion 110 and end portion 112 are configured so that they may lay flat against the soft tissue surrounding the bone for ease of insertion into an incision. End portion 112 further includes a condyle silhouette. The condyle silhouette includes a curved edge 123 that generally mimics a silhouette of a distal condyle of a femur.

As shown in FIGS. 1A and 1B, extension arm 108 protrudes from end portion 112 of plate body 102 from a lateral side of end portion 112 proximate to second end 106 of plate body. Extension arm 108 is separated from plate body 102 by a space. In some examples, and as depicted in FIGS. 1A and 1B, extension arm 108 is generally parallel to end portion 112 of plate body 102. Extension arm 108 and plate body 102 may be formed monolithically or extension arm 108 may be attached to plate body 102. Extension arm 108 is fixed to end portion 112 of plate body 102 via a transition portion 116. Transition portion 116 is a curved extension of end portion 112 that protrudes externally from plate body 102. Extension arm 108 further includes a gripping portion 118 adjacent to transition portion 116. Gripping portion 118 includes a neck portion 120 having a first curved surface 122 and a second curved surface 124 opposite the first curved surface and separated by a distance from the first curved surface. First and second curved surfaces 122, 124 are both concave, defining neck portion 120 along gripping portion 118 of extension arm 108. Gripping portion 118 of extension arm 108 is configured to grasp around a bone. In some examples, first curved surface 122 may be shaped to complement a shape of an inferior pole of a patella bone. Extension arm 108 further includes a free end 126.

End portion 112 of plate body 102 may also include indicia adjacent second end 106 of plate body 102. As shown in FIG. 1B, such indicia is in the form of a linear marking 128 adjacent to extension arm 108 on an outer side of plate body 102. Linear marking 128 is a horizontal line, i.e., a line extending across a width of the plate body. In some examples, linear marking is in a range from 10 mm to 20 mm from second end 106, and parallel to second end 106. For instance, and as shown in FIG. 1B, linear marking 128 may be positioned approximately 14 mm from second end 106 of plate body 102. Linear marking 128 may be used as a reference to aid in the identification of resection levels along a bone to which the resection template is applied. In particular, when template 100 is positioned on a femur, linear marking 128 provides a user with an indication of a location of the inferior pole of the patella relative to the template. Described another way, first curved surface 122 of gripping portion 118 of extension arm 108 is contoured to receive a bone such as the patella. Linear marking 128 is then used as a guide to locate the inferior pole of the patella which may be used as a reference to locate an accurate resection level along the femur. Although not shown in FIGS. 1A and 1B, template 100 may also include measuring indicia along elongate portion 110 of plate body 102. Such indicia may indicate a distance from another location on the template, such as linear marking 128. FIG. 1C provides an example of plate body 102 of bone template 100 engaging soft tissue around a femur 12 and extension arm 108 gripping an end of a patella 14.

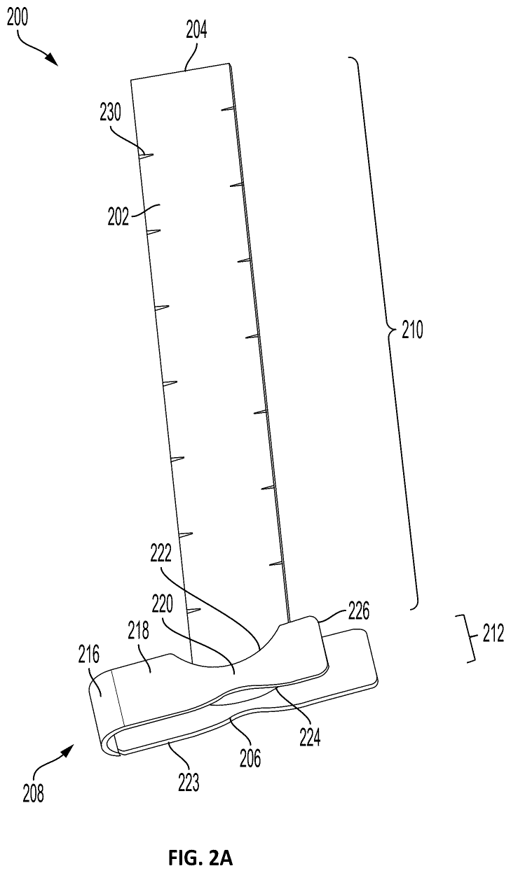

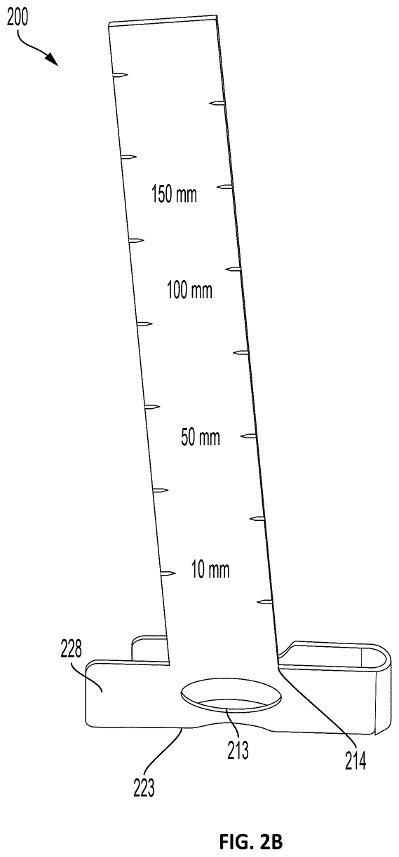

In another embodiment, bone resection template 200, as shown in FIGS. 2A and 2B, includes a plate body 202 having an elongate portion 210, an end portion 212, and an extension arm 208. The design of bone resection template 200 of FIGS. 2A and 2B is similar to that of FIGS. 1A and 1B. Bone resection template 200 of FIGS. 2A and 2B, includes measuring indicia along elongate portion 210 of plate body 202. Measuring indicia may include markings along elongate portion 210 as well as a plurality of notches 230 along one or both lateral edges of the elongate portion 210. Measuring indicia may include numeral indications of distance, such as millimeters of distance from linear marking 228. Notches 230 may be spaced evenly along the edges of elongate portion 210. Notches 230 may have a shape and size that varies from the notches shown in FIGS. 2A and 2B. Bone resection template 200 of FIGS. 2A and 2B also includes an end portion 212 of plate body 202 having a length extending from a second end 206 of plate body 202 to transition location 214 located at a transition point between elongate portion 210 and end portion 212 of plate body 202. The length of end portion 212 extending from second end 206 of plate body 202 to transition location 214 may be approximately equal to a length of extension arm 208 at free end 226 extending in a direction parallel to the length of end portion 212. Further, a width of end portion 212 extending in the medial and lateral direction may also be approximately equal to that of extension arm 208. In other examples, length and width dimensions of the end portion and the extension arm may vary with respect to each other. End portion 212 may also include an oblong cut out 213 on the outer side of plate body 202. Oblong cut out 213 may allow for visualization and access to the inferior pole of patella.

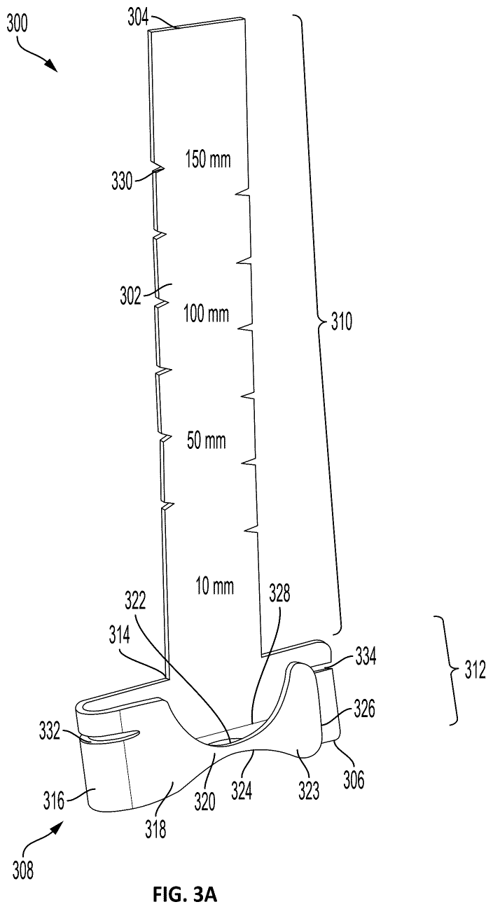

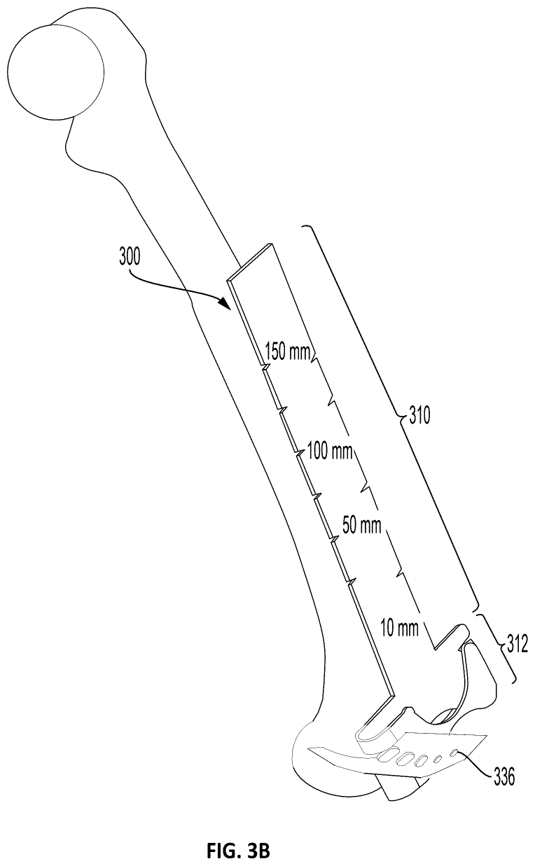

In yet another embodiment, bone resection template 300, as shown in FIG. 3A, includes a plate body 302 having an elongate portion 310, an end portion 312, and an extension arm 308. The design of bone resection template 300 of FIG. 3A is similar to that of FIGS. 2A and 2B. However, bone resection template 300 of FIG. 3, includes a slot 332 on extension arm 308 of plate body 302 and a separate slot 334 on end portion 312 of plate body 302. Slot 332 on extension arm 308 is positioned on transition portion 316 and extends to gripping portion 318 of extension arm 308 on one side and may extend partially onto end portion 312 on an opposite side. Slot 332 is configured to allow a reference plate to be attached to plate body 302 to provide an additional reference point. For example, as shown in FIG. 3B, slot 332 may be used as a reference to a medial epicondyle using an angel wing 336 or a similar device received in slot 332. In addition, linear marking 328 is located on inner side of plate body 302. Linear marking 328 is positioned on inner side of plate body 302 to allow bone resection template 300 to provide accurate measurements with reference to a length of the bone As described in greater detail in the methods of using the template, bone resection template 300 may be positioned directly against a bone surface during use.

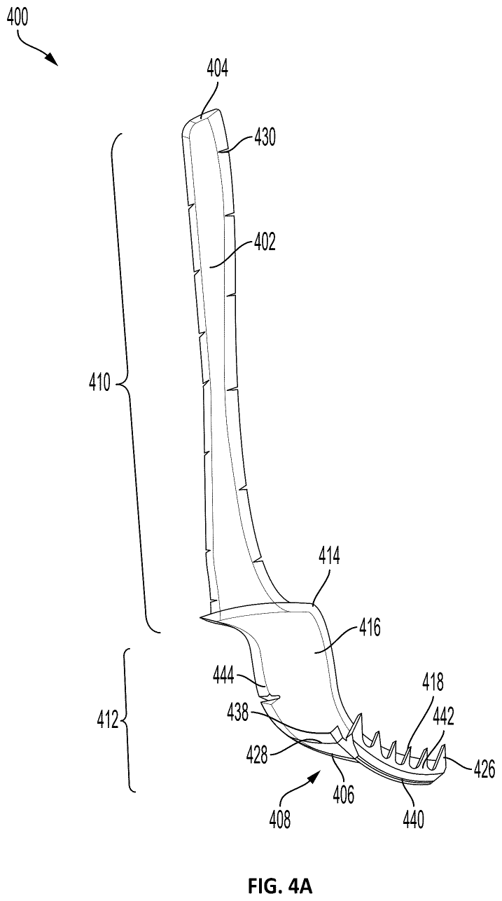

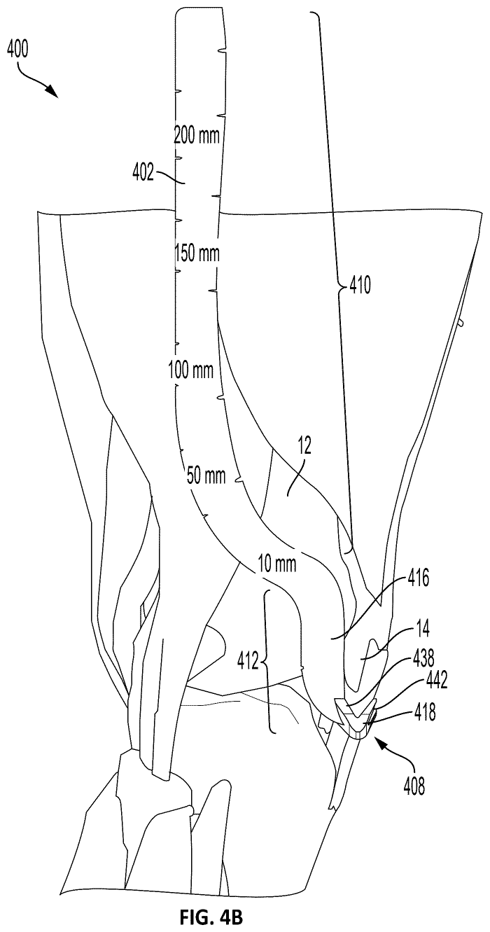

In yet another embodiment, bone resection template 400, as shown in FIG. 4A includes a plate body 402 having a measuring or elongate portion 410, an end portion 412 and an extension arm 408. End portion 412 of plate body 402 includes a transition portion 416 that connects elongate portion 410 of plate body 402 to extension arm 408. elongate portion 410 extends from a first end 404 to a transition location 414 located at a transition point located between elongate portion 410 and transition portion 416. Elongate portion 410 includes measuring indicia along elongate portion 410 of plate body 402. Measuring indicia may include markings along elongate portion 410 as well as a plurality of notches 430 along one or both lateral edges of the elongate portion 410. Measuring indicia may include numeral indications of distance, such as millimeters of distance from linear marking 428. Notches 430 may be spaced evenly along the edges of elongate portion 410. Notches 430 may have a shape and size that varies from the notches shown in FIGS. 4A and 4B.

Extension arm 408 includes a base portion 438 and a gripping portion 418 that are positioned relative to each other to form a v-shaped trough within extension arm 408 along a second end 406 of plate body 402. Extension arm 408 extends from transition portion 416 of plate body 402, transition portion 416 having a free end 426. In variations, a shape of the trough may be u-shaped or may have another shape, such as any shape that provides room to receive a bone thereon. Base portion 438 extends directly from transition portion 416 of end portion 412 of plate body 402. Gripping portion 418 includes a curved surface 440 across a width of gripping portion 418, the curved surface having a plurality of teeth 442 protruding along upper side of curved surface 440 of gripping portion 418. Plurality of teeth 442 are used to grip around a bone or a soft tissue near the bone. Transition portion 416 includes a linear marking 428 adjacent to second end 406 of plate body 402 proximate to extension arm 408. Linear marking 428 may be used as a reference to aid in the identification of resection levels along a bone to which template 400 is applied. In particular, when template 400 is positioned on a femur, linear marking 428 provides a user with an indication of a location of the inferior pole of the patella relative to the template. Transition portion 416 of plate body may also include at least one slot 444 to house an additional reference plate relative to the template.

FIG. 4B provides an example of elongate portion 410 of bone resection template 400 engaging the femur 12 and the extension arm 408 gripping end of the patella 14. For example, knee surgery incisions are usually performed on the medial side, thus, it is preferred to have the elongate portion on the medial side of extension arm. Template 400 as shown in FIG. 4B includes only a medial arm having an elongate portion. This allows for the insertion of only a medial arm of bone resection template into the incision. In this case, separate bone resection templates having individual medial and lateral arms are required for left and right femurs. In a variation, it is contemplated to have a bone resection template that is the same as template 400 but having an opposite shape such that it is adapted for use on a lateral side of the leg.

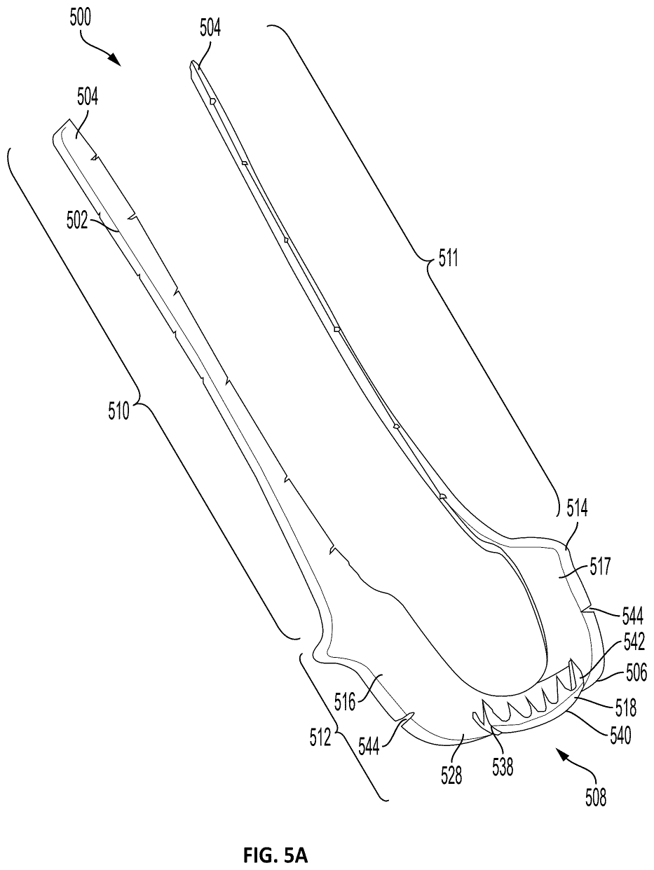

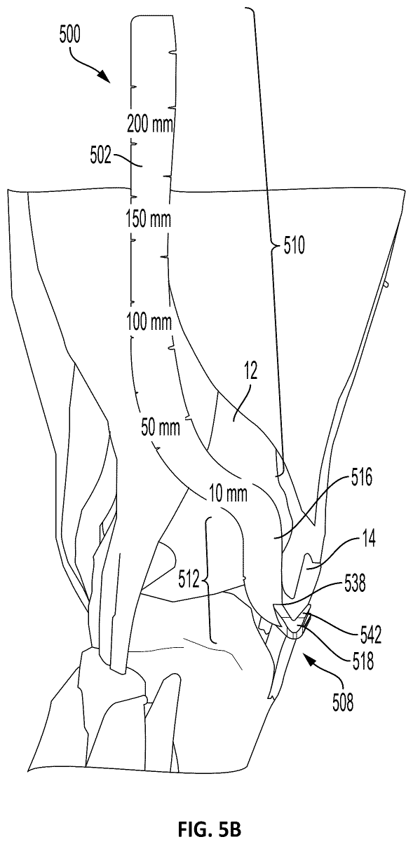

In yet another embodiment, bone resection template 500, as shown in FIG. 5A includes a plate body 502 having a first elongate portion 510, a second elongate portion 511, an end portion 512 that connects the first and second elongate portions, and an extension arm 508 extending from end portion 512. The design of bone resection template 500 of FIG. 5A is similar to that of FIG. 4A. The bone resection template 500 of FIG. 5 includes an extension arm 508 protruding from end portion 512 of plate body 502. End portion 512 includes a first transition portion 516 and a second transition portion 517. The first and second transition portions connect first elongate portion 510 to second elongate portion 511. In some examples, the first and second transition portions are positioned as to form a u-shaped end portion. Each portion of the plate body has a surface contoured to better conform with the curvature of the femur and sit flush on the bone. As the plate body wraps around the femur, the measuring indicia along each elongate portion are not obscured by the patella. With some examples of bone resection template 500, the same bone resection template can be used for both left and right femurs as there are medial and lateral arms extending from respective ends of the end portion. In other examples, bone resection template 500 may be shaped for use on a left or right femur. FIG. 5B provides an example of elongate portion 510 of bone template 500 engaging soft tissue around femur 12 and extension arm 508 gripping an end of a patella 14.

Templates 400 and 500 include a contoured plate body designed to curve around the femur bone. In some embodiments, the contoured plate body may be designed to be anatomically specific, thereby ensuring accurate curvature to better conform to the femur bone and sit flush on the bone surface. The templates shown have been designed using a range of patient data stored in a database, such patient data being usable to create universal instruments for groupings of patients having similar sized bones or patients with other shared anatomical characteristics.

The bone resection templates as described above may be made of a biocompatible material, such as a polymer e.g., reinforced polymer composites; a titanium alloy e.g., Ti6Al4V; stainless steel e.g., 316L, 17-4 PH SS; or other metals and metal alloys, such as Ti and CoCr. Additionally, templates may be manufactured via one or more of sheet metal forming, stamping, bending, laser cutting, and additive manufacturing processes such as one or more of the processes described in U.S. Pat. Nos. 4,863,538, 5,017,753, 5,076,869, 4,944,817, 7,537,664, 10,614,176, 11,534,307 and 11,737,880, hereby incorporated by reference herein.

In another aspect, the present disclosure relates to a device for measuring a bone cut. The device includes an elongate member extending from a first end portion to a second end portion opposite first end portion. The elongate member includes a measurement portion having indicia that are used to indicate a dimension to guide resection level along a bone. The indicia include notches along the measurement portion. Device also includes a gripping member extending from the second end portion of the elongate member. The gripping member has a width dimension that extends laterally away from a central longitudinal axis of the measurement portion of the elongate member. The gripping member is separated from the elongate member by a distance. The gripping member is shaped to be positionable against a bone surface. The gripping member includes a concave edge that grasps the bone surface. The bone is at least partially located between the gripping member and the elongate member. The second end portion includes a curved edge positionable against a second bone surface.

In one embodiment, the gripping member is connected to the elongate member via a curved member that extends from one end of the elongate member to one end of the gripping member. The gripping member has a free end.

In another embodiment, the gripping member is connected to the elongate member via a curved member extending from opposite ends of the gripping member. The gripping member may also include a concave edge having a plurality of pointed protrusions extending from concave edge positionable against a bone surface.

In further embodiments, a secondary bone resection template may be joined to the bone resection template to measure and determine desired bone cuts along a proximal tibia.

In another aspect, the present disclosure relates to surgical kits for use during a distal femoral resection or a large segmental bone replacement surgery. A kit may include any of the various resection templates described herein in any combination and in any quantity. For example, in some embodiments, two or more of bone resection templates 100, 200, 300, 400, 500 may be combined in a kit with a reference plate, or variations of reference plate. In other embodiments, a kit may include multiple bone resection templates of the same type, e.g., 100, 200, 300, 400, 500, and may optionally include a reference plate, such as angel wing 336. Bone resection template 400 may include two separate bone resection templates, a medial template and a lateral template. In any of the above embodiments, two or more of the templates included in a kit may be different sizes. In any one of the above embodiments, a kit may include two or more reference plates. Such reference plates may be the same or may be different.

Further, a kit may also include various insertion instrumentation, such as grippers, jaws, cutting tools, reamers, etc., and other instrumentation used to resect the femoral bone and implant the femoral components. In still further embodiments, any of the contemplated kits may also include other implant components such as tibial components, patellar components, etc. The contemplated kits allow an operator to have freedom to choose a template best suited for a patient and where additional instrumentation is included, allows for the selection of an implant best suited for use on a prepared distal femur while also complementing and fitting anatomy in a joint. A kit may be advantageous when a procedure requires both robotic bone cuts and manual bone cuts. In other examples, a kit may be well suited for revision procedures, where an extent of bone loss may vary greatly among patients. For such surgical applications, a kit including an array of bone resection templates is advantageous in that a range of patient bone decay conditions may be accommodated with an appropriate bone resection template from the kit.

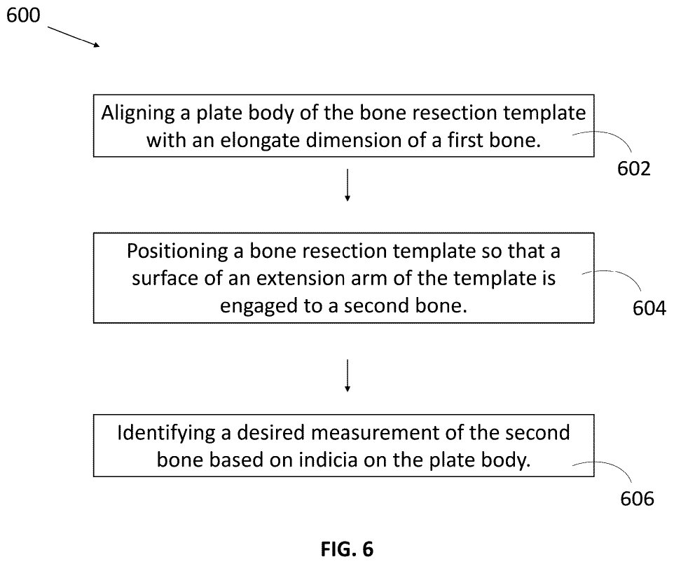

In yet another aspect, the present disclosure relates to a method of determining an extent of a desired bone cut from a length of a bone or measuring the resection level along a bone, for example, measuring the resection level along the femur for revision of the distal femur during a total knee replacement. One embodiment of such method is method 600 shown in FIG. 6. Method 600 of determining an extent of a bone cut includes a step 602 of aligning a plate body of the bone resection template with an orientation of elongate dimension of a first bone, a step 604 of positioning a contoured surface of an extension arm of a bone resection template plate body on a second bone, and a step 606 of identifying a desired measurement of the first bone based on indicia on the plate body. The desired measurement represents a length of a portion of the first bone from an end of the first bone proximate the extension arm to the indicia. To prepare for the performance of the above steps, an incision must be created so that the bone resection template plate body may be inserted into the body of the patient. In some examples, the plate body may be inserted through a previous incision made during a total knee arthroplasty. Such incisions may include, but are not limited to, medial parapatellar, mid vast and sub vastus approaches. In other examples, a new incision may be formed to access the body.

In some embodiments, a method of determining an extent of a desired bone cut utilizes bone resection template 100 shown in FIGS. 1A and 1B. In this method, step 602 involves aligning the plate body with an orientation of an elongate dimension of a first bone, e.g., femur, so that the plate body is aligned with the first bone. The first bone, e.g., a femur, is located adjacent to the second bone, e.g., a patella, that the extension arm is grasped around. The plate body may lay against the surrounding soft tissue of the bone and may be positioned as shown in FIG. 1C. With bone resection template 100 as shown in FIGS. 1A and 1B, plate body 102 has a narrower width along elongate portion 110 of plate body 102 and a greater width along end portion 112 of plate body 102 proximal to extension arm 108. The narrower width of plate body 102 along elongate portion 110 allows bone template 100 to be easily inserted into small revision incisions during surgery. Then, plate body 102 is aligned with the elongate dimension of the first bone.

Additionally, end portion 112 of plate body 102 proximate extension arm 108 may have a curved edge 123 shaped similar to a distal condyle of the femur. This curved edge 123 allows for a first reference measurement using the first bone by aligning curved edge 123 with the curved edge of the first bone, e.g., condyles of the femur. Utilization of curved edge 123 as a first reference measurement to align with the femoral condyle silhouette may be particularly advantageous in oncology cases. The bone resection template can be used to guide the resection of a bone to a level that can be reproduced by the available implants. When utilizing the silhouette feature, the bone resection template can be placed against the first bone so that the silhouette of the condyle on the end portion of the bone template coincides with the distal condyle of the femur.

In methods utilizing bone template 100, as shown in FIG. 1C, the bone template may be placed on the soft tissue surrounding the femur for ease of insertion after an incision into the body. After the bone template is positioned against the soft tissue, the bone template may be moved upward as to allow engagement with the patella.

Step 604 of positioning the contoured surface on the extension arm of the bone resection template onto the second bone may include positioning a concave edge or a first curved surface 122 of the extension arm 108 against a corresponding surface of the second bone, e.g., patella when used to measure femur. In such step, first curved surface 122 is configured to grasp around an end of second bone such that extension arm 108 of bone resection template 100 grips the second bone, as shown in FIG. 1C. Grasping around the end of the second bone will cause the second bone gripped by the template to align with a linear marking 128 located on plate body 102. Linear marking 128 allows for the determination of a joint line associated with the first bone. Linear marking 128 also assists in the positioning of extension arm 108 around the second bone end. Template 100 may be positioned with an inner surface of plate body 102 facing the first bone, e.g., femur since linear marking 128 may be included on an outer surface of plate body 102.

In some examples of bone resection template 100, and indeed in other contemplated bone resection templates, the linear marking may be located approximately 14 mm from the end of the plate body proximate the extension arm so that a joint line, e.g., a joint line between the femur and the tibia, is located approximately 14 mm from the linear marking. Thus, when the template is used on the femur, the linear marking may reference the inferior pole of the patella or the bottom tip of the patella, which is near the femur. This is beneficial in that if the bone template is used to measure resection levels along the femur, reference to the patella avoids the need to reference the femur, which may be in a deteriorated state or have a shape that is malformed due to bone loss or disease. A distal femoral condyle in such condition cannot be used as accurate reference to measure the resection level along the femur. The bone resection template of the present disclosure is configured to utilize the inferior pole of the patella as a proxy for the femur to avoid this issue.

Step 606 involves identifying a desired measurement of the first bone, e.g., femur, based on indicia along the plate body to determine at least one resection level along the first bone in reference to the second bone grasped by the extension arm. Indicia on template 100 may be notches 230 or other indicia as contemplated by the present disclosure included on plate body 102. Such indicia indicate a dimension relative to a reference location on template 100. In template 100, such reference location is linear marking 128. Thus, the indicia also provide measurement information related to an interior pole of the patella and the joint line between the femur and the tibia.

The markings or indicia along the plate body are then read at the appropriate resection level. If used to resect a femur, the indicia are read at a resection level with consideration of whether the femoral implant stem being used is with or without extra-cortical porous-coated body sections. An anterior cortex of the first bone, such as the femur, is marked with a Bovie or a similar device to indicate resection level along the first bone in preparation for the resection before the implant is inserted. When resecting the femur, the patella is important as a reference because a length of femoral resection and the details of the implant must be considered in order to establish proper patellar tracking. The joint line between the femur and the tibia is also important as a reference to recreate a preoperative leg length. Thus, patellar tracking, tibial cut, and leg length must be considered when making a femoral resection. In some embodiments, the method may continue after measurements are taken with the template. In such embodiments, a distal replacement implant may be implanted in the patient. Further, in some examples, a femoral implant stem with or without an extra-cortical porous-coated body section may be connected to an extension piece or a distal femoral replacement implant.

In some embodiments, a method of determining an extent of a desired bone cut utilizes bone resection template 200 or 300, shown in FIGS. 2A and 2B and FIGS. 3A and 3B, respectively. Unless otherwise stated, the steps of using bone resection templates 200, 300 are the same as those described for bone resection template 100. When performing step 602, templates 200, 300 may be positioned with an outer surface of plate body 202, 302 facing the first bone, e.g., femur since the indicia to measure a resection length may be included on an inner surface. Additionally, for template 300, placing the linear marking 328 on inner side of bone reference template 300 allows for easier placement of bone template flat against first bone as opposed to surrounding body tissue. Otherwise, the engagement of the contoured surface 222, 322 on the extension arm of the bone resection templates 200 and 300 onto the second bone is similar to that of bone resection template 100.

In methods utilizing bone template 300, the template may be placed directly on the surface of the femur. To facilitate such placement, after incision into the body, the patella and the soft tissue surrounding the femur are moved laterally to gain exposure to the bone surface. Then, the template is positioned to lay flat against the bone surface. After the bone template is positioned on the femur, the patella is moved laterally back toward its previous position to engage the extension arm of the bone template. By placing the template against the bone surface, the method need not rely on a position of the soft tissue around the bone as a guideline. Additionally, placing the bone template against the bone mitigates the risk of any parallax error.

For methods utilizing bone template 300, in addition to the notches 330 along plate body 302 a slot 332 is located along extension arm 308 of bone resection template 300 so that a reference plate such as an angel wing 336 may be received therein. For example, slot 332 may be used as a reference to medial epicondyle using an angel wing 336 template that is inserted into slot 332. In some examples, slot 332 is located 28 mm above the second end 306 of plate body 302 proximate to extension arm 308. Angel wing 336 is inserted horizontally into slot 332. The step 606 of identifying a desired measurement of the first bone based on indicia along the plate body of template 300 is similar to that of templates 400 and 500.

In some embodiments, a method 600 of determining an extent of a desired bone cut utilizes bone resection templates 400 or 500, shown in FIGS. 4A and 4B and FIGS. 5A and 5B, respectively. Unless otherwise stated, the steps of using bone resection templates 400, 500 are the same as those described for bone resection template 100. In use, step 602 includes gripping portion 418 which grips onto the second bone using a plurality of teeth 442 and a trough adjacent to the teeth. Such step is performed in a similar manner with bone resection template 500.

To align the plate body 402, 502 with an orientation of an elongate dimension of a first bone as part of step 604, elongate portions 410, 510 are aligned with the first bone. Plate body 402, 502 includes elongate portions 410, 510 contoured to allow for plate body 402, 502 to lay directly against the bone surface. When using template 500, a user may choose one elongate portion 510, 511 over another based on which one may work best based on the specific patient's anatomy and the specific surgical circumstances. In template 500, the first and second elongate portions are both contoured to conform to the femur bone.

In methods utilizing bone templates 400 and 500, the template may be placed directly on the surface of the femur. To facilitate such placement, after incision into the body, the patella and the soft tissue surrounding the femur are moved laterally to gain exposure to the bone surface. Then, the template is positioned to lay flat against the bone surface. After the bone template is positioned on the femur, the patella is moved laterally back toward its previous position to engage the extension arm of the bone template. The bone template may be contoured to wrap around the femur and the measuring indicia may extend along the side of the femur bone as to not be obscured by the patella.

As many bone revision surgeries and cases involving severe bone loss do not have a pristine condyle or other bone structure it is difficult to determine the joint line between the tibia and the femur and the patella position after implantation. Thus, grasping the patella using the extension arm and using the interior pole of the patella as a reference guide allows for a more accurate bone resection level. The patella is typically in a pristine or at least non-deteriorated condition during total knee arthroplasty procedures. Bone revision cases typically have significant bone loss in the condyles such that the condyles cannot be referenced, but the patella is available as a reference. Further as bone tumor surgeries typically have pristine condyles, the bone template of the present disclosure allows for the condyle to also be used as an additional reference. Thus, the bone template of the present disclosure is versatile in that it can be used in both tumor and revision cases.

It is to be understood that the disclosure set forth herein includes any possible combinations of the particular features set forth above, whether specifically disclosed herein or not. For example, where a particular feature is disclosed in the context of a particular aspect, arrangement, configuration, or arrangement, that feature may also be used, to the extent possible, in combination with and/or in the context of other particular aspects, arrangements, configurations, and arrangements of the technology, and in the technology generally.

It should be noted that any of the bone resection templates and methods disclosed herein may be used in conjunction with robotic technology. For example, any of the templates described herein may be used with robotic surgical systems to determine the extent of the bone cut and cut the bone. Further, any or all of the steps described in the method of determining the extent of the desired bone cut from length of the bone of the present disclosure may be performed using a robotic system.

Although the disclosure herein has been described with reference to particular embodiments, it is to be understood that these embodiments are merely illustrative of the principles and applications of the present disclosure. For example, features described in relation to one particular embodiment may be combined with features of other embodiments described herein. It is therefore to be understood that numerous modifications may be made to the illustrative embodiments and that other arrangements may be devised without departing from the spirit and scope of the present disclosure as defined in the appended claims.

1. A bone resection template comprising:
a plate body having a length extending from a first end to a second end opposite the first end; and
an extension arm protruding from an end portion of the plate body proximate the second end, the extension arm including a portion with a curved surface contoured to receive a bone therein such that when the bone is received on the curved surface portion, the plate body is on one side of the bone.

2. The bone template of claim 1, wherein the plate body comprises an elongate portion and the end portion, the end portion being wider than the elongate portion, and the extension arm extending from a lateral side of the end portion.

3. The bone template of claim 2, wherein the extension arm further comprises a transition portion and a gripping portion, the transition portion defining a space that separates the gripping portion of the extension arm from the plate body and the gripping portion including a neck portion between the transition portion and a free end of the extension arm.

4. The bone template of claim 3, wherein the neck portion of the extension arm is defined by a first curved surface and a second curved surface opposite the first curved surface, and the first curved surface and the second curved surface are concave.

5. The bone template of claim 1, wherein the plate body includes a plurality of indicia along a direction between the first end and the second end, the indicia indicative of a dimension from an end of a bone measured by the bone template.

6. The bone template of claim 1, wherein the extension arm includes at least one slot.

7. The bone template of claim 2, wherein the end portion of the plate body includes indicia representative of a location of a bone-gripping edge of the extension arm along a direction of the length of the plate body.

8. The bone template of claim 1, wherein the plate body comprises an elongate portion and a transition portion that connects the elongate portion of the plate body to the extension arm.

9. The bone template of claim 8, wherein the extension arm extends from a single end of the plate body, the extension arm having a length from the plate body to a free end.

10. The bone template of claim 1, wherein the extension arm further comprises a base portion and a gripping portion positioned to form a trough within the extension arm.

11. The bone template of claim 10, wherein the gripping portion comprises a curved surface, the curved surface having a plurality of teeth protruding from the curved surface of the gripping portion.

12. The bone template of claim 10, wherein the elongate portion of the plate body includes a plurality of indicia along a direction of the length of the plate body.

13. The bone template of claim 1, wherein the plate body further comprises a first elongate portion and a second elongate portion, the extension arm separating the first and second elongate portions.

14. A device for measuring a bone cut, the device comprising:
an elongate member extending from a first end portion to a second end portion opposite the first end portion, the elongate member including a measurement portion with indicia indicating a dimension based on a size of the device; and
a gripping member extending from the second end portion, the gripping member having a width dimension such that the gripping member includes a portion that extends laterally away from a central longitudinal axis of the measurement portion of the elongate member,
wherein the gripping member is shaped to be positionable against a bone surface such that the bone is at least partially in between the gripping member and the elongate member.

15. The device of claim 14, wherein the gripping member includes a concave edge positionable against the bone surface.

16. The device of claim 14, wherein the second end portion includes a curved edge positionable against a second bone surface.

17. The device of claim 14, wherein the indicia include notches along the measurement portion.

18. The device of claim 14, wherein the elongate member and the gripping member are connected via a curved member on the gripping member that extends from the gripping member to the elongate member.

19. The device of claim 18, wherein the gripping member includes a plurality of pointed protrusions extending from a concave edge of the gripping member.

20. The device of claim 18, wherein the elongate member includes a second measuring portion and a second curved member.
